(19) United States
(12) Patent Application Publication
(10) Pub. No.: US 2004/0081619 A1
(43) Pub. Date: Apr. 29, 2004

(54) Method of prophylaxis against large myocardial infarctions

(76) Inventor: Leonard Bell, Woodbridge, CT (US)

Correspondence Address: Mark Farber, C/O Alexion Pharmaceuticals, Inc., 352 Knotter Drive, Cheshire, CT, 06410, US

(21) Appl. No.: 10/690,190

(22) Filed: Oct. 21, 2003

Related U.S. Application Data
(62) Division of application No. 10/047,608, filed on Jan. 14, 2002.
(60) Provisional application No. 60/262,540, filed on Jan. 18, 2001.

(51) Int. Cl. A61K 49/00

(52) U.S. Cl. 424/9.2

(57) ABSTRACT

Methods of determining the effectiveness of anti-inflammatory compounds in reducing incidence of myocardial infarction are described. Methods of prophylaxis against myocardial infarctions which exhibit CK-MB levels greater than about 50 nano-grams/ml in a subject are also provided.

### BACKGROUND

1. Technical Field

This disclosure relates to methods of determining the effectiveness of anti-inflammatory compounds in reducing incidence of myocardial infarction. Methods of prophylaxis against myocardial infarctions which exhibit CK-MB levels greater than about 50 nano-grams/ml in a subject are also described.

2. Background of Related Art

Coronary artery disease is often characterized by lesions or occlusions in the coronary arteries which may result in inadequate blood flow to the myocardium, or myocardial ischemia, which is typically responsible for such complications as angina pectoris, necrosis of cardiac tissue (myocardial infarction), and sudden death. In some cases, coronary artery disease may be treated by the use of drugs and by modifications in behavior and diet. In other cases, dilatation of coronary arteries may be achieved by such procedures as angioplasty, laser ablation, atherectomy, catheterization, and intravascular stents.

For certain patients, coronary artery bypass grafting (CABG) is the preferred form of treatment to relieve symptoms and often increase life expectancy. CABG consists of direct anastomosis of a vessel segment to one or more of the coronary arteries. For example, a reversed segment of the saphenous vein may be grafted at one end of the ascending aorta as an arterial blood source and at the other end to a coronary artery at a point beyond the arterial occlusion. Alternatively, the internal mammary artery is located in the thoracic cavity adjacent the sternum and is likewise suitable for grafting to a coronary artery, such as the left anterior descending artery.

During the CABG surgery, the heart is usually stopped from beating, to facilitate the anastomosis procedures. While the heart is not beating, extracorporeal circulation of the blood supports most of the patient's body (excluding the heart and, to some extent, the lungs). A cardiopulmonary bypass (CPB) machine receives deoxygenated blood from the patient's body, adds oxygen and various nutrients to the blood, and pumps the oxygenated blood back into the patient's body.

Although CABG surgery has substantially improved the therapeutic outcome of patients with advanced myocardial ischemia, the recovery period may be often traumatic to the patient with significant attendant risks. For example, it is known that CPB elicits a systemic inflammatory response that causes tissue injury and contributes to significant perioperative and long-term clinical morbidity. During CPB, exposure of blood to bioincompatible surfaces of the extracorporeal circuit, as well as tissue ischemia and reperfusion associated with the procedure, induces the activation of several major humoral pathways of inflammation. Clinical manifestations attributed to this systemic inflammatory response may include myocardial injury which may manifest as myocardial infarction (heart cell death) or as severe ventricular dysfunction requiring circulatory assist.

Techniques to measure damage to the heart, using blood chemistry are known in the art. When heart cells die, certain enzymes that are normally kept inside viable cells are released into the circulating blood. One such enzyme is creatine kinase (CK), which catalyzes the reversible transfer of a phosphate group from ATP to creatine. It exists as a dimer composed of two subunits commonly identified as the M-subunit and the B-subunit. CK-MB is associated with myocardial infarction, and is present in serum in only trace concentrations in the absence of such an episode (other isoenzymes CKMM and CK-BB are found in skeletal muscle and brain cells). Appearance of CK-MB isoenzyme in serum is, therefore, indicative of myocardial infarction.

Therefore, it is known in the art that, if a drug can reduce CK-MB levels in blood during and/or after CABG surgery involving CPB, the reduction in blood CK-MB levels indicates that the drug helped protect the heart against cell death and tissue damage.

Fitch et al., Pharmacology and Biological Efficacy of a Recombinant, Humanized, Single-Chain Antibody C5 Complement Inhibitor in Patients Undergoing Coronary Artery Bypass Graft Surgery With Cardiopulmonary Bypass (Circulation, 1999; 100:2499.), disclose that h5G1.1-scFv, a recombinant single-chain antibody C5 inhibitor, proved to be a potent inhibitor of systemic complement activation, inhibiting both complement-dependent hemolytic activity and, more importantly, the generation of the proinflammatory activation product C5-9 and C5b-9 in patients undergoing CPB. Fitch et al. further disclose that the potent complement inhibitory and anti-inflammatory activities of h5G1.1-scFv were associated with significant reductions in post-operative CK-MB release, new cognitive deficits, and blood loss. The potent inhibitory and anti-inflammatory effects of h5G1.1-scFv were associated with significant reductions in postoperative myocardial injury. In addition, Fitch measured CD11 b on activated neutrophils and monocytes, and reported that in doses sufficient to completely block hemolytic activity and soluble C5b-9 generation (e.g. 1.0 and 2.0 mg/kg), h5G1.1-scFv significantly attenuated peak leukocyte CD11b expression compared with the placebo. Nonetheless, Fitch et al. (citing Gray et al., Circulation, 1982:66:1185-1'189; Calliff et al. J.AmColl Cardiol, 1998:31:241-251; Abdelmeguid et al. Circulation, 1995:91:2733-2741; and Kong et al. JAMA, 1997:277:461-466) state that “there does not appear to be a threshold effect, but rather, it is apparent that the greater the release of CK-MB, the greater the subsequent morbidity, cost, and mortality, . . . [and that] it is likely that significant reductions in postoperative myocardial injury might be associated with improved outcomes”.

However, no method of detecting and/or differentiating inflammatory damage from traumatic damage in patients having undergone CABG involving CPB based on postoperative CK-MB peak levels in the blood exists in the art. Hence, no method of testing the efficacy of an anti-inflammatory drug by monitoring CK-MB peak levels in such patients exists. Accordingly, no method of prophylaxis against large myocardial infarction (which more often result in mortality, e.g., such as those which exhibit CK-MB levels greater than about 50 nano-grams/ml) is known in the art. Further, the relative utility of anti-inflammatory drugs to limit larger, as opposed to smaller, post-CABG myocardial infarctions is not known.

It would be advantageous to provide a method of testing the efficacy of an anti-inflammatory drug by monitoring CK-MB peak levels in patients having undergone CABG involving CPB. It would be of further advantage to provide a method of prophylaxis against large myocardial infarctions as indicated by peak CK-MB levels of about 50 nano-grams/ml or more.

### SUMMARY

A method of determining effectiveness of an anti-inflammatory compound in reducing incidence of post-CABG myocardial infarction has now surprisingly been found. This method includes administering an anti-inflammatory compound to a subject group including at least one patient undergoing a procedure involving cardiopulmonary bypass; comparing incidence of infarctions in the subject group to incidence of infarctions in a control sample of patients for a given peak CK-MB level in the blood (such as, for example, greater than 50 nano-grams/ml) in both groups; wherein a decrease in the incidence of infarctions in the subject group indicates effectiveness of the compound.

In another embodiment a method of prophylaxis against myocardial infarctions which exhibit peak CK-MB levels greater than about 50 nano-grams/ml in a subject is provided. This method includes administering to the subject undergoing a procedure involving cardiopulmonary bypass an effective myocardial infarction reducing amount of an anti-inflammatory compound.

### BRIEF DECRIPTIPON OF THE DRAWINGS

FIG. 1 is a graph summarizing the reduction of incidence of myocardial infarction which exhibit various CK-MB levels that was provided by an anti-C5 antibody, namely h5G1.1-scFv in a controlled, randomized clinical test of patients undergoing CABG with CPB.

FIG. 2 is a graphical presentation of the data of Table One showing the reduction in peak CK-MB values resulting from the use of h5G1.1-scFv.

### DETAILED DESCRIPTION OF PREFERRED EMBODIMENTS

The methods of determining effectiveness of an anti-inflammatory compound in reducing incidence of myocardial infarction in accordance with this disclosure includes the step of administering an anti-inflammatory compound to a subject group including at least one patient undergoing a procedure which involves cardiopulmonary bypass. Such procedures include, but are not limited to CABG and heart transplant. The level of CK-MB in the patients' blood is measured and the incidence of myocardial infarctions which exhibit peak CK-MB levels greater than about 50 nano-grams/ml is determined. The incidence of such infarctions in the subject group is then compared to the incidence of infarctions exhibiting a comparable level of CK-MB in a control sample of patients. A decrease in the incidence of infarctions in the subject group indicates effectiveness of the compound.

Anti-inflammatory compounds which can be evaluated by the methods described herein include non-steroidal anti-inflammatory actives or drugs (NSAIDS). The NSAIDS can be selected from the following categories: propionic acid derivatives; acetic acid derivatives; fenamic acid derivatives; biphenylcarboxylic acid derivatives; and oxicams. All of these NSAIDS are fully described in the U.S. Pat. No. 4,985,459 to Sunshine et al., issued Jan. 15, 1991, incorporated by reference herein. Most preferred are the propionic NSAIDS including, but not limited to aspirin, acetaminophen, ibuprofen, naproxen, benoxaprofen, flurbiprofen, fenoprofen, fenbufen, ketoprofen, indoprofen, pirprofen, carprofen, oxaprozin, pranoprofen, miroprofen, tioxaprofen, suprofen, alminoprofen, tiaprofenic acid, fluprofen and bucloxic acid. Another useful class of anti-inflammatory compounds include inhibitors of cyclooxygenase-1 (COX-1) and inhibitors of cyclooxygenase-2 (COX-2). Also useful are the steroidal anti-inflammatory drugs including hydrocortisone and the like. Particularly useful are anti-inflammatory compounds which reduce neutrophil activation or monocyte activation by greater than about 30%.

Preferred anti-inflammatory compounds are compounds which bind to or otherwise block the generation and/or activity of complement components. A specific class of such compounds which are particularly useful are antibodies specific to a human complement component.

The complement system acts in conjunction with other immunological systems of the body to defend against intrusion of cellular and viral pathogens. There are at least 25 complement proteins, which are found as a complex collection of plasma proteins and membrane cofactors. The plasma proteins make up about 10% of the globulins in vertebrate serum. Complement components achieve their immune defensive functions by interacting in a series of intricate but precise enzymatic cleavage and membrane binding events. The resulting complement cascade leads to the production of products with opsonic, immunoregulatory, and lytic functions. A concise summary of the biologic activities associated with complement activation is provided, for example, In The Merck Manual, 16′ Edition.

The complement cascade progresses via the classical pathway or the alternative pathway. These pathways share many components, and while they differ in their initial steps, they converge and share the same “terminal complement” components (C5 through C9) responsible for the activation and destruction of target cells. The classical complement pathway is typically initiated by antibody recognition of and binding to an antigenic site on a target cell. The alternative pathway is usually antibody independent, and can be initiated by certain molecules on pathogen surfaces. Additionally, the lectin pathway is typically initiated with binding of mannose-binding lectin (MBL) to high mannose substrates. These pathways converge at the point where complement component C3 is cleaved by an active protease (which is different in each pathway) to yield C3a and C3b. Other pathways activating complement attack can act later in the sequence of events leading to various aspects of complement function.

C3a is an anaphylatoxin (see discussion below). C3b binds to bacterial and other cells, as well as to certain viruses and immune complexes, and tags them for removal from the circulation. (C3b in this role is known as opsonin.) The opsonic function of C3b is generally considered to be the most important anti-infective action of the complement system. Patients with genetic lesions that block C3b function are prone to infection by a broad variety of pathogenic organisms, while patients with lesions later in the complement cascade sequence, i.e., patients with lesions that block C5 functions, are found to be more prone only to Neisseria infection, and then only somewhat more prone (Fearon, in Intensive Review of Internal Medicine, 2^{nd} Ed. Fanta and Minaker, eds. Brigham and Women's and Beth Israel Hospitals, 1983).

C3b also forms a complex with other components unique to each pathway to form classical or alternative C5 convertase, which cleaves C5 into C5a and C5b. C3 is thus regarded as the central protein in the complement reaction sequence since it is essential to both the alternative and classical pathways (Wurzner, et al., Complement Inflamm. 8:328-340, 1991). This property of C3b is regulated by the serum protease Factor I, which acts on C3b to produce iC3b. While still functional as opsonin, iC3b cannot form an active C5 convertase.

C5a is another anaphylatoxin (see discussion below). C5b combines with C6, C7, and C8 to form the C5b-8 complex at the surface of the target cell. Upon binding of several C9 molecules, the membrane attack complex (MAC, C5b-9, terminal complement complex—TCC) is formed. When sufficient numbers of MACs insert into target cell membranes the openings they create (MAC pores) mediate rapid osmotic lysis of the target cells. Lower, non-lytic concentrations of MACs can produce other effects. In particular, membrane insertion of small numbers of the C5b-9 complexes into endothelial cells and platelets can cause deleterious cell activation. In some cases activation may precede cell lysis.

As mentioned above, C3a and C5a are anaphylatoxins. These activated complement components can trigger mast cell degranulation, which releases histamine and other mediators of inflammation, resulting in smooth muscle contraction, increased vascular permeability, leukocyte activation, and other inflammatory phenomena including cellular proliferation resulting in hypercellularity. C5a also functions as a chemotactic peptide that serves to attract pro-inflammatory granulocytes to the site of complement activation.

Any compounds which bind to or otherwise block the generation and/or activity of any of the human complement components, such as, for example, antibodies specific to a human complement component are useful herein. Some compounds include 1) antibodies directed against complement components C-1, C-2, C-3, C-4, C-5, C-6, C-7, C-8, C-9, Factor D, Factor B, Factor P, MBL, MASP-1, AND MASP-2 and 2) naturally occurring or soluble forms of complement inhibitory compounds such as CR1, LEXCR1, MCP, DAF, CD59, Factor H, cobra venom factor, FUT-175, y bind protein, complestatin, and K76 COOH. Suitable compounds for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. Particularly useful complement inhibitors are compounds which reduce the generation of C5a and/or C5b-9 by greater than about 30%. A particularly useful anti-C5 antibody is h5G1.1-scFv. Methods for the preparation of h5G1.1-scFv are described in U.S. patent application Ser. No. 08/487,283 filed Jun. 7, 1995 now U.S. Pat. No. ______ and “Inhibition of Complement Activity by Humanized Anti-C5 Antibody and Single Chain Fv”, Thomas et al., Molecular Immunology, Vol. 33, No. 17/18, pages 1389-1401, 1996, the disclosures of which are incorporated herein in their entirety by this reference.

The following non-limiting example is included to illustrate the present invention but is not intended to limit the scope thereof.

### EXAMPLE

### Randomized, Double-Blind, Placebo Controlled Study of the Effect of h5G1.1-scFv on Total Mortality and Adverse Cardiovascular Ischemic Outcomes in Patients Undergoing Cardiopulmonary Bypass

A randomized, multi-center, double-blind, placebo-controlled study was conducted of h5G1.1-scFv administered to patients at moderately increased risk of adverse post-operative ischemic events undergoing CPB as part CABG.

The study population consisted of individuals who elected to undergo non-emergent coronary-artery bypass graft (CABG) surgery, without valve surgery, which required the use of a cardiopulmonary bypass (CPB) machine. There were approximately 270 patients for each of the three treatment arms.

Patients were randomized to receive one of the following three treatment combinations: i) Bolus 2.0 mg/kg h5G1.1-scFv followed by 0.05 mg/kg/hr h5G1.1-scFv for 24 hours; ii) Bolus 2.0 mg/kg h5G1.1-scFv; and iii) Placebo. The h5G1.1-scFv or matching placebo was provided as a solution for injection in 30 ml vials with a concentration of 2 mg/ml. Patients received the bolus of study medication ten (10) minutes before the initiation of cardiopulmonary bypass via a unique line. The drug was not to be combined with other medication given via this route. The infusion began immediately following bolus administration, and continued for 24 hours at a constant drip rate.

Patients were evaluated at an initial screening visit, which occurred within 14 days prior to the first administration of study medication. Blood pressure and heart rate were recorded every 15 minutes throughout the intraoperative period, beginning at the induction of anesthesia.

For purposes of CK-MB measurements, intra- and post-operative blood draws were performed at 4, 8, 16, 20, 24, 30 and 36 hours post-CPB. Additionally, the post operative day (POD) 2 CK-MB draw was at 48 hours post-CPB. There was a 30 minute window for each of these blood draws except for those drawn in the OR, which were exact. The POD 4 CK-MB draw was collected with routine blood draws. Measurements of CK-MB were made using a microparticle enzyme immunoassay that is commercially available under the tradename AxSYM system from Abbott Laboratories, (Abbott Park, Ill.).

The study was conducted in accordance with standard operating procedures designed to ensure adherence to Good Clinical Practice (GCP) as required by the following: Directive 91/507/EEC: The Rules Governing Medicinal Products in the European Community; Code of Federal Regulations dealing with clinical studies (21 CFR including parts 50 and 56 concerning informed consent and IRB regulations); and Declaration of Helsinki, concerning medical research in humans (“Recommendations Guiding Physicians in Biomedical Research Involving Human Subjects,” Helsinki 1964, amended Tokyo 1975, Venice 1983, Hong Kong 1989 and Somerset West 1996). Note for Guidance on Good Clinical Practice in valid version approved by CPMP in July 1996. International Conference on Harmonisation; Good Clinical Practice: Consolidated Guideline; Notice of Availability, in Federal Register, May 9, 1997.

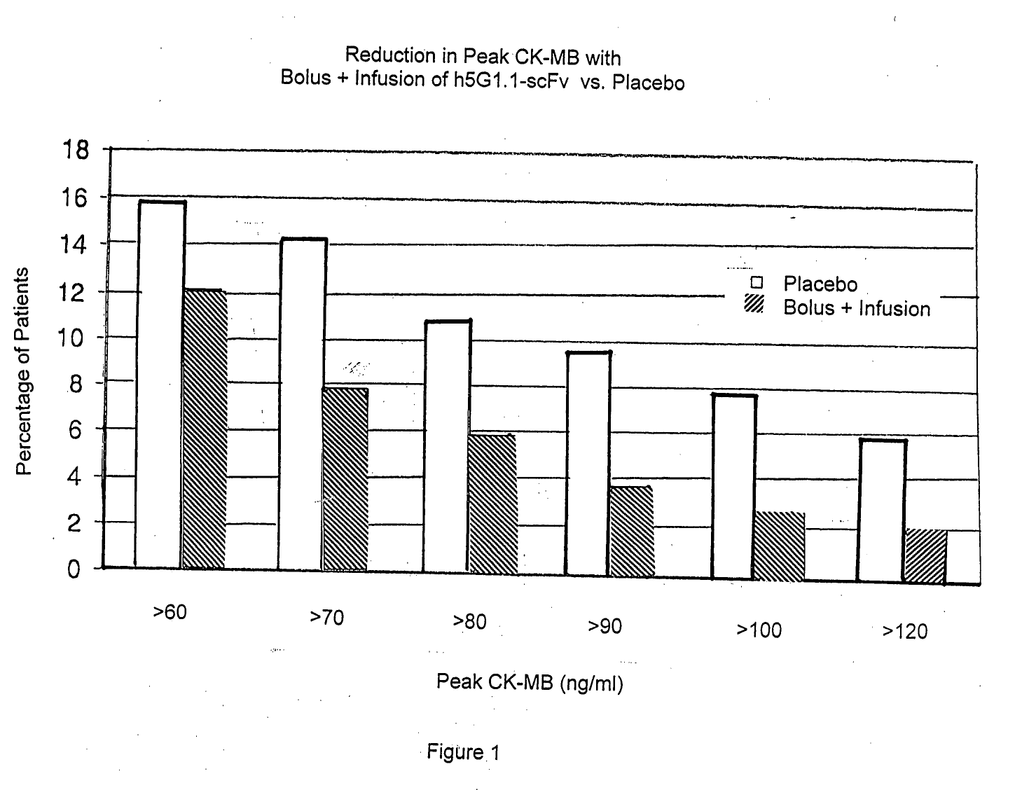

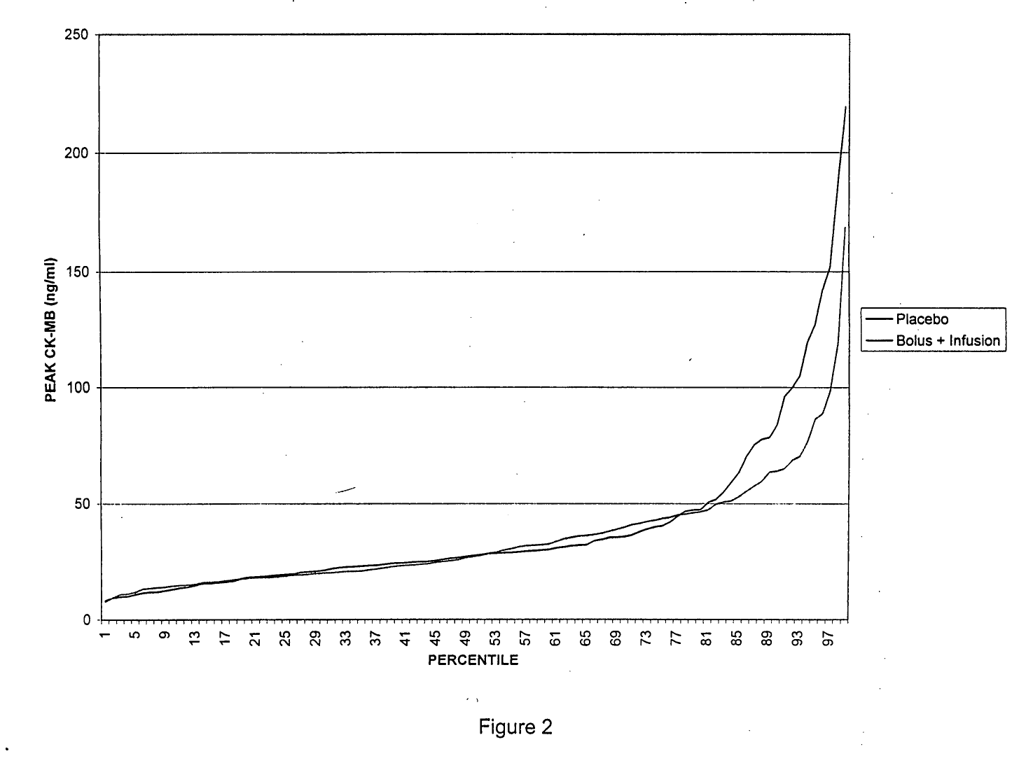

The distribution of peak CK-MB levels for each patent group was subjected to conventional statistical analysis to calculate percentiles. The results are summarized in FIGS. 1 and 2. As seen in FIG. 1, a significant decrease in the incidence of myocardial infarctions which exhibit CK-MB levels of each of >60 ng/ml, >70 ng/ml, >80 ng/ml, >90 ng/ml, >110 ng/ml and, >120 ng/ml was provided by h5G1.1-scFv. As seen in FIG. 2, the effectiveness of administering the anti-inflammatory compound surprisingly is observed to be significant only in patients experiencing myocardial infarction which exhibits a peak CK-MB value of greater than about 50 ng/ml.

In another aspect, a novel method for testing the anti-inflammatory drug efficacy and formulation of endpoints in CABG clinical trials has been discovered. Specifically, by using the methods disclosed herein endpoints in CABG trials with myocardial infarction defined, in part, by CK-MB peak levels of >50, >60, >70, >80, >90, >100 or >120 can be effectively utilized to evaluate anti-inflammatory drugs.

Because the anti-inflammatory compound evaluated using the methods herein may be determined to reduce the incidence of myocardial infarctions of such severity to exhibit a CK-MB level of greater than 50 ng/ml, in another aspect, this disclosure contemplates a method of prophylaxis against myocardial infarctions which exhibit CK-MB levels greater than about 50 nano-grams/ml in a subject. This method includes administering to the subject undergoing a procedure which involves CPB an effective myocardial infarction reducing amount of an anti-inflammatory compound. Ascertaining what amount constitutes an effective myocardial infarction reducing amount of the anti-inflammatory compound can be ascertained using the novel screening procedure described hereinabove, or by any technique known to those skilled in the art. The dosage of the anti-inflammatory compound that constitutes an effective myocardial infarction reducing amount will depend on a number of factors, including, for example, the specific anti-inflammatory compound selected and its method of operation. However, typically the anti-inflammatory compound can be administered in an amount ranging from about 0.01 mg/kg to about 20.0 mg/kg, preferably from about 0.10 mg/kg to about 10.0 mg/kg.

Any anti-inflammatory compound evaluated using the methods herein and determined to reduce the incidence of myocardial infarctions may be used in the present method of prophylaxis. Any compounds which bind to or otherwise block the generation and/or activity of any of the human complement components, such as, for example, antibodies specific to a human complement component are useful for prophylaxis. Some compounds include 1) antibodies directed against complement components C-1, C-2, C-3, C-4, C-5, C-6, C-7, C-8, C-9, Factor D, Factor B, Factor P, MBL, MASP-1, AND MASP-2 and 2) naturally occurring or soluble forms of complement inhibitory compounds such as CR1, LEX-CR1, MCP, DAF, CD59, Factor H, cobra venom factor, FUT-175, y bind protein, complestatin, and K76 COOH, Suitable compounds for use herein are antibodies that reduce, directly or indirectly, the conversion of complement component C5 into complement components C5a and C5b. One class of useful antibodies are those having at least one antibody-antigen binding site and exhibiting specific binding to human complement component C5, wherein the specific binding is targeted to the alpha chain of human complement component C5. Such an antibody 1) inhibits complement activation in a human body fluid; 2) inhibits the binding of purified human complement component C5 to either human complement component C3 or human complement component C4; and 3) does not specifically bind to the human complement activation product for C5a. A particularly useful anti-C5 antibody is h5G1.1-scFv.

Although preferred and other embodiments of the invention have been described herein, further embodiments may be perceived by those skilled in the art without departing from the scope of the invention as defined by the following claims.

What is claimed:

1. The method of determining effectiveness of an anti-inflammatory compound in reducing incidence of myocardial infarction comprising:
administering the compound to a subject group comprising at least one patient undergoing a procedure involving cardiopulmonary bypass; and
comparing incidence of infarctions in the subject group to incidence of infarctions in a control sample of patients when the peak level of CK-MB in the blood is greater than 50 nano-grams/ml in both groups;
wherein a decrease in the incidence of infarctions in the subject group indicates effectiveness of the compound.

2. The method of claim 1, wherein the procedure is CABG surgery.

3. The method of claim 1, wherein the CK-MB level is greater than about 60 nano-grams/ml.

4. The method of claim 1, wherein the CK-MB level is greater than about 70 nano-grams/ml.

5. The method of claim 1, wherein the CK-MB level is greater than about 80 nano-grams/ml.

6. The method of claim 1, wherein the CK-MB level is greater than about 90 nano-grams/ml.

7. The method of claim 1, wherein the CK-MB level is greater than about 100 nano-grams/ml.

8. The method of claim 1, wherein the CK-MB level is greater than about 120 nano-grams/ml.

9. The method of claim 1, wherein the anti-inflammatory compound is a complement inhibitor.

10. The method of claim 9, wherein the complement inhibitor is selected from the group consisting of a) antibodies directed against complement components C-1, C-2, C-3, C-4, C-5, C-6, C-7, C-8, C-9, Factor D, Factor B, Factor P, MBL, MASP-1, or MASP-2; and b) naturally occurring or soluble forms of CR1, LEXCR1, MCP, DAF, CD59, Factor H, cobra venom factor, FUT-175, y bind protein, complestatin and K76 COOH.

11. The method of claim 9, wherein the antibody directly or indirectly reduces the conversion of complement component C5 into complement components C5a and C5b.

12. The method of claim 11, wherein the anti-C5 antibody is an antibody comprising at least one antibody-antigen binding site, said antibody exhibiting specific binding to human complement component C5, said specific binding being targeted to the alpha chain of human complement component C5, wherein the antibody 1) inhibits complement activation in a human body fluid; 2) inhibits the binding of purified human complement component C5 to either human complement component C3 or human complement component C4; and 3) does not specifically bind to the human complement activation product for C5a.

13. The method of claim 9, wherein the complement inhibitor specifically binds to a component forming the C5b-9 complex.
